(19) United States
(12) Patent Application Publication
(10) Pub. No.: US 2024/0063390 A1
(43) Pub. Date: Feb. 22, 2024

(54) COVALENT ORGANIC FRAMEWORK AND ENERGY STORAGE DEVICE

(71) Applicant: City University of Hong Kong, Kowloon (HK)

(72) Inventors: Qichun Zhang, Kowloon (HK); Chun Sing Lee, Kowloon (HK); Shen Xu, Kowloon (HK); Chenchen Wang, Kowloon (HK)

(21) Appl. No.: 17/888,935

(22) Filed: Aug. 16, 2022

(51) Int. Cl. H01M 4/60; H01M 10/0525; H01M 10/04; C07F 1/04; C07F 1/02

(52) U.S. Cl. CPC H01M 4/60; H01M 10/0525; H01M 10/0436; C07F 1/04; C07F 1/02; H01M 2004/028

(57) ABSTRACT

A covalent organic framework includes a plurality of aromatic moieties each linked by at least one thioether linkage, and its preparation method. An energy storage device includes a cell with an electrode including the covalent organic framework.

### TECHNICAL FIELD

The invention relates to a covalent organic framework. The invention also relates to an energy storage device.

### BACKGROUND

Lithium-ion batteries (LIBs) have been widely commercialized for decades for their remarkable performance, but they suffer from high costs and safety problems. Sodium-ion batteries (SIBs) are one of the alternatives to LIBs. However, obtaining high-performance cathode materials is the main issue that restrict the improvement of the capacity of SIBs.

Comparing with inorganic electrode materials, organic electrode materials are renewable, designable for specific function, and have higher theoretical gravimetric capacities. Some organic small molecules with active groups have shown excellent device performance when utilized as a cathode material for SIBs. Nevertheless, they exhibit obvious drops on the capacities from the second cycle due to their solubility in electrolytes, which is disadvantageous for long-term stability of batteries.

### SUMMARY

In a first aspect, there is provided a covalent organic framework, comprising: a plurality of aromatic moieties each linked by at least one thioether linkage. Preferably, each of the plurality of aromatic moieties is linked by two thioether linkages.

Optionally, each of the plurality of aromatic moieties comprises at least one carbonyl group, and preferably two carbonyl groups.

Optionally, each of the plurality of aromatic moieties is a benzoquinone moiety. The covalent organic framework may include a plurality of dithioether-linked benzoquinone moieties.

In a second aspect, there is provided an electrode comprising a covalent organic framework, which may be the covalent organic framework in the first aspect. The electrode may further comprise a conductive material, a binder, and a current collector.

In a third aspect, there is provided an energy storage device, comprising a cell with an electrode comprising a covalent organic framework, which may be the covalent organic framework in the first aspect.

Optionally, the covalent organic framework further comprises a plurality of redox active sites for facilitating diffusion of ions of the energy storage device. The plurality of redox active sites may comprise a sulfur atom of the thioether linkages.

Optionally, each of the plurality of aromatic moieties comprises at least one carbonyl group, and the plurality of redox active sites comprise the carbonyl groups.

Optionally, the electrode is a cathode. The cell may be rechargeable and may take the form of a coin cell. Optionally, the cell may comprise a sodium-ion battery or a lithium-ion battery.

In a fourth aspect, there is provided a method of preparing a covalent organic framework, comprising the steps of: mixing an aromatic compound and a thiol to form a mixture, degassing the mixture, and heating the degassed mixture.

Optionally, the aromatic compound comprises a quinone, such as a halogen-substituted quinone. For example, the halogen-substituted quinone is 2,3,5,6-tetrachloro-1,4-benzoquinone or 2,3,5,6-tetrafluoro-1,4-benzoquinone.

Optionally, the thiol is an aromatic thiol, e.g., 1,2,3,4,5,6-benzenehexathiol.

Optionally, the step of mixing comprises mixing a base and a solvent with the aromatic compound and the thiol to form the mixture, the base comprising any one of sodium carbonate, potassium carbonate, cesium carbonate, sodium hydroxide, sodium methoxide, and N,N-diethylethanamine, and the solvent comprising any one of 1,2-dichlorobenzene, n-butanol, 1,4-dioxane, mesitylene, N-methylpyrrolidone, N,N-dimethylacetamide, or a mixture thereof.

Optionally, the method further includes sonicating the mixture to form a homogeneous mixture.

Optionally, the step of degassing comprises subjecting the mixture to at least one freeze-pump-thaw cycle and preferably three freeze-pump-thaw cycles.

In a fifth aspect, there is provided a method of fabricating an energy storage device, comprising the steps of: assembling components of the energy storage device within a larger case and a smaller case in a predetermined order, and packing the larger case and the smaller case together with a pressure of about 0.1 kPa to about 1.5 kPa.

Other features and aspects of the invention will become apparent by consideration of the detailed description and accompanying drawings. Any feature(s) described herein in relation to one aspect or embodiment may be combined with any other feature(s) described herein in relation to any other aspect or embodiment as appropriate and applicable.

### BRIEF DESCRIPTION OF THE DRAWINGS

Embodiments of the invention will now be described, by way of example, with reference to the accompanying drawings in which:

FIG. 1 is a schematic diagram of the synthesis of a covalent organic framework (referred to as BHT-BQ-COF) in accordance with one embodiment of the invention;

FIG. 2 is a schematic exploded view of an energy storage device in accordance with one embodiment of the invention;

FIG. 3 shows the Fourier transform infrared (FT-IR) spectra of the precursors of BHT-BQ-COF, model compound, and BHT-BQ-COF;

FIG. 4 is a thermogravimetric curve of BHT-BQ-COF;

FIG. 5 shows the cyclic voltammetry (CV) curves of a BHT-BQ-COF-based electrode in a sodium-ion battery for the initial three cycles scanned at 0.1 mV s⁻¹ with a voltage range of 0.1 V to 3.5 V;

FIG. 6 shows the voltage-capacity curves of the BHT-BQ-COF-based electrode in the sodium-ion battery at the first, fifth, and tenth cycles;

FIG. 7A is a graph of capacity and coulombic efficiency against cycle number of the BHT-BQ-COF-based electrode in the sodium-ion battery at the current density of 200 mA g⁻¹;

FIG. 7B is a graph of capacity and coulombic efficiency against cycle number of the BHT-BQ-COF-based electrode in the sodium-ion battery at the current density of 400 mA g⁻¹;

FIG. 8 is a graph of capacity and coulombic efficiency against cycle number of the BHT-BQ-COF-based electrode in the sodium-ion battery at a wide current density range of 100 mA g⁻¹ to 5000 mA g⁻¹ with a voltage range of 0.1 V to 3.5 V; and

FIG. 9 show the electrochemical impedance spectroscopy (EIS) curves of the sodium-ion battery Na//BHT-BQ-COF-based electrode at different charge and discharge states with a wide frequency range from 100 kHz to 100 mHz.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. Terms of 25 degree, such as “substantially” or “about” are understood by those of ordinary skill to refer to reasonable ranges outside of the given value, for example, general tolerances associated with manufacturing, assembly, testing, and use of the described embodiments.

In one embodiment, there is provided a covalent organic framework (COF) that includes a plurality of aromatic moieties each linked by at least one thioether linkage. In a preferred embodiment, the COF includes a dithioether linkage. As used herein, “covalent organic framework” refers to an organic crystalline porous material that integrates molecular building blocks (i.e., the aromatic moieties) connected by covalent linkages and integrated into a periodic structure, which may be extended into two or three dimensions. In one embodiment, the COF may have a porous yet rigid structure.

As will be appreciated by those skilled in the art, due to the purely covalently bonded and metal-free structures of COFs, they generally exhibit excellent chemical stability in organic solvents and withstand harsh conditions (e.g., acidic and basic conditions) to maintain their ordered structures and crystallinity. As such, the COF in the present embodiment may be used in gas separation and storage, heterogeneous catalysis, chemical sensing, luminescence, electronic devices, drug delivery, and energy storage and conversion.

As used herein, an “aromatic moiety” includes any of substituted or unsubstituted C₃₋₈ cycloalkyl, substituted or unsubstituted C₃₋₈ cycloalkenyl, substituted or unsubstituted 3 to 8 membered heterocycloalkyl, substituted or unsubstituted 3 to 8 membered heterocycloalkenyl, substituted or unsubstituted C₆₋₁₀ aryl, substituted or unsubstituted C₇₋₁₁ aralkyl, substituted or unsubstituted heteroaryl having 5 to 10 carbon atoms or heteroatoms. Preferably, the aromatic moiety includes at least one carbonyl group, e.g., a benzoquinone moiety with two carbonyl groups.

A “thioether linkage” refers to the covalent linkage connecting the building blocks including a R—S—R′ bond where R is one of the aromatic moieties and R′ is the adjacent aromatic moiety or a functional group that is connected to the adjacent aromatic moiety. A “dithioether linkage” means two thioether linkages, i.e., there are two said R—S—R′ bonds connected between the pair of aromatic moieties. The functional group for R′ may be any of substituted or unsubstituted C₃₋₈ cycloalkyl, substituted or unsubstituted C₃₋₈ cycloalkenyl, substituted or unsubstituted 3 to 8 membered heterocycloalkyl, substituted or unsubstituted 3 to 8 membered heterocycloalkenyl, substituted or unsubstituted C₆₋₁₀ aryl, substituted or unsubstituted C₇₋₁₁ aralkyl, substituted or unsubstituted heteroaryl having 5 to 10 carbon atoms or heteroatoms. Preferably, the linkage between the aromatic moieties is a benzenehexathiol group.

In one embodiment, the COF may be prepared from an aromatic compound and a thiol. For example, the aromatic compound may be a quinone, more preferably a halogen-substituted quinone. The thiol may be an aromatic thiol having at least one S—H group for forming at least one thioether linkage in the COF.

The method of preparing the COF may including the steps of: mixing the aromatic compound and the thiol to form a mixture, degassing the mixture, and heating the degassed mixture.

The step of mixing may involve a condensation or a cross-coupling reaction between the aromatic compound and the thiol. For example, the step of mixing may be performed in a reaction vessel specifically designed for use in air-sensitive chemistry, e.g., a Schlenk flask or tube. The step of mixing may further include mixing a base and a solvent with the aromatic compound and the thiol to form the mixture. The base may be any one of sodium carbonate, potassium carbonate, cesium carbonate, sodium hydroxide, sodium methoxide, and N,N-diethylethanamine. The solvent may be any one of 1,2-dichlorobenzene, n-butanol, 1,4-dioxane, mesitylene, N-methylpyrrolidone, N,N-dimethylacetamide, or a mixture thereof.

After the step of mixing and before the step of degassing, the method may further include sonicating the mixture to form a homogeneous mixture. For example, the mixture may be sonicated for about 1 min to about 30 min, about 3 min to about 15 min, or about 5 min to about 12 min.

As will be appreciated by those skilled in the art, during the step of degassing, dissolved gasses (e.g., oxygen, carbon dioxide, etc.) that may otherwise impede the chemical reactions involving sensitive reagents, interfere with spectroscopic measurements, or induce unwanted bubble formation are removed from the mixture.

In one embodiment, the step of degassing includes subjecting the mixture to at least one freeze-pump-thaw cycle, which involves (i) freezing the mixture, (ii) applying vacuum to the frozen mixture, then (iii) sealing and warming the mixture. Specifically, the mixture in the reaction vessel is first sealed to prevent a flow of inert gas into the vessel whilst being frozen in, e.g., liquid nitrogen or dry ice for the mixture to reach a temperature below about 80 K. Once the mixture in the vessel has frozen, the vessel can be unsealed while being kept under vacuum for about 1 min to about 20 min, about 3 min to about 15 min, or about 5 min to about 10 min, allowing evacuation of the headspace. The vessel may also remain submerged in liquid nitrogen or dry ice during vacuum application. Next, the vessel may be sealed again under vacuum and removed from the liquid nitrogen or dry ice to allow the mixture to reach room temperature. This may in addition involve the use of a heat source to speed up warming of the mixture. As the mixture thaws, the dissolved gasses escape into the headspace. This process may be repeated for 10 times, 5 times, or 3 times to remove the undesired dissolved gasses.

Alternatively or additionally, the step of degassing may involve heating, ultrasonic agitation, chemical removal of gasses, substitution with inert gas by bubbling, etc.

The method may then proceed to the step of heating the degassed mixture, e.g., at about 30° C. to about 300° C., about 40° C. to about 200° C., or about 60° C. to about 130° C., for about 24 hours to about 120 hours, about 48 hours to about 96 hours, or about 72 hours. The heated mixture may then be allowed to cool down to room temperature, before being filtered, washed (e.g., with water, dimethylformamide, tetrahydrofuran, and dichloromethane) and dried to obtain the COF. Without wishing to be bound by theories, it is believed that the synthesis in the present embodiment achieves a product yield of about 65% to about 85%.

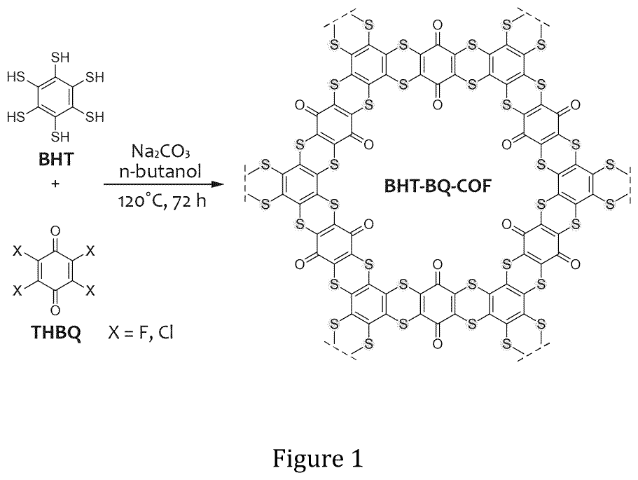

In a preferred embodiment, the COF is BHT-BQ-COF, which is constructed of dithioether-linked benzoquinone moieties. FIG. 1 shows the synthesis of BHT-BQ-COF which involves a cross-coupling reaction between a halogen-substituted quinone and an aromatic thiol. In the illustrated embodiment, the aromatic thiol is 1,2,3,4,5,6-benzenehexathiol (BHT), and the halogen-substituted quinone is a tetra-halogen-substituted benzoquinone (THBQ), e.g., 2,3,5,6-tetrafluoro-1,4-benzoquinone (TFBQ) or 2,3,5,6-tetrachloro-1,4-benzoquinone (TCBQ). They are mixed with sodium carbonate as the base and n-butanol as the solvent in the step of mixing, before being degassed and heated at 120° C. for 72 hours.

As discussed above, the COF may be used in energy storage applications. More specifically, the COF may be used as an electrode (e.g., a cathode) in an energy storage device, e.g., a sodium-ion battery or a lithium-ion battery that may be rechargeable. The redox active sites provided by the sulfur atoms of the thioether linkages and the carbonyl groups of the quinone moieties, as well as the porous and rigid structure, facilitate diffusion of ions (e.g., ion insertion and extraction) of the device and provide excellent stability of the electrode in the electrolyte, thus maximizing the capacity and providing excellent reversibility for the device.

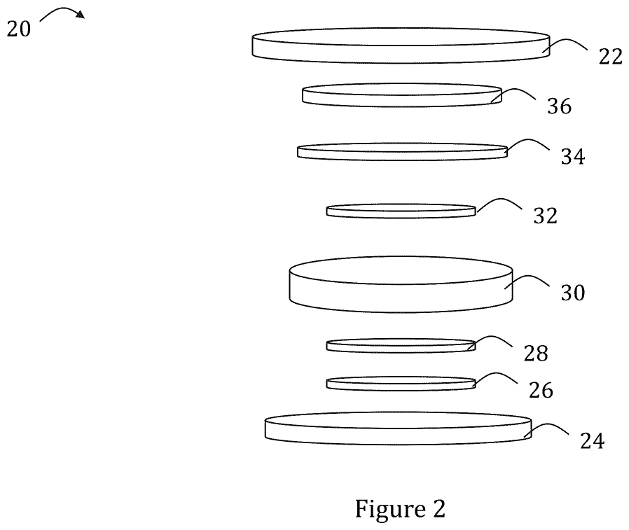

FIG. 2 shows an example energy storage device 20 that utilizes a COF as a cathode. The illustrated energy storage device 20 takes the form of a coin cell. The coin cell 20 includes a casing with a larger case 22 (e.g., the positive side) and a smaller case 24 (e.g., the negative case). The components within the casing include, assembled in the following order: an o-ring 26 adjacent the smaller case 24, a spring 28, a spacer 30, an anode 32, a separator 34 and an electrolyte, and a cathode 36 adjacent the larger case 22 having a cast film closer to the anode 32. The spacer 30 may be a stainless steel spacer. The anode 32 may be a metal piece, such as a metal foil, a metal disk, etc. The spacer 30 and the anode 32 may be first coupled together before the assembly of the coin cell 20. The separator may be placed closer to the anode than the spacer, and may be a glass fiber. The electrolyte may be a liquid, gel, or solid electrolyte, e.g., sodium bis(fluorosulfonyl)imide (NaFSI), sodium bis(trifluoromethanesulfonyl)imide (NaTFSI), or sodium hexafluorophosphate (NaPF₆). The electrolyte may further include a solvent, e.g., diglyme or 1-methyl-1-propylpyrrolidinium bis(trifluoromethanesulfonyl)imide (py13TFSI). Preferably, the electrolyte has a concentration of about 0.5 M to about 4.0 M of the above material in the solvent.

The cathode 36 may include the COF, a conductive material, a binder, and a current collector. The conductive material may include conductive carbon, such as Super P, Ketjen Black, or carbon nanotube, etc. The binder may include polyvinylidene fluoride (PVDF). The current collector may be a copper or aluminum foil. The cathode 36 is preferably centered as much as possible with the anode 32 to avoid uneven current densities.

In one embodiment, the preparation of the cathode 36 includes the steps of: mixing the COF, the conductive material, the binder, and a solvent (e.g., N-methyl-2-pyrrolidone (NMP)) to form a slurry, pasting the slurry onto the current collector, and drying the assembly. Preferably, the mass ratio of the COF, the conductive material, and the binder in the slurry is about 7:2:1 to about 3:6:1. The step of drying may be performed in a vacuum oven at about 30° C. to about 150° C., about 50° C. to about 130° C., or about 60° C. to about 120° C., for about 3 h to about 48 h, about 5 h to about 36 h, or about 6 h to about 24 h.

In one embodiment, the preparation of the energy storage device 20 includes the step of: assembling the components of the device 20 in the above-mentioned order, and packing the larger case 22 and the smaller case 24 together with a pressure of about 0.1 kPa to about 1.5 kPa, about 0.5 kPa to about 1.0 kPa, or about 0.75 kPa to about 0.85 kPa. The step of assembling may include the following steps in sequential order: pressing the anode 32 against the spacer 30, placing and pressing the o-ring 26 onto the smaller case 24, placing the spring 28 on top and then the spacer 30 and anode 32 (with the anode 32 facing upwards), placing the separator 34 on top as centered as possible, dropping the electrolyte (e.g., about 100 μL to about 300 μL) onto the separator 34, placing the cathode 36 on top (with the cast film facing the anode 32), and placing the larger case 22 on top.

Hereinafter, the present invention is described more specifically by way of examples, but the present invention is not limited thereto.

Unless otherwise specifically provided, all tests herein are conducted at standard conditions which include a room and testing temperature of 25° C., sea level (1 atm.) pressure, pH 7, and all measurements are made in metric units. Furthermore, all percentages, ratios, etc. herein are by weight, unless specifically indicated otherwise. It is understood that unless otherwise specifically noted, the materials compounds, chemicals, etc. described herein are typically commodity items and/or industry-standard items available from a variety of suppliers worldwide. All the reagents and solvents are used without further purification unless otherwise specified. The n-butanol, 1,2-dichlorobenzene, and 1,4-dioxane are purchased as extra dry grade with water lower than 50 ppm.

### Example 1

### Synthesis of BHT-BQ-COF

A Schleck tube was charged with 1,2,3,4,5,6-benzenehexathiol (BHT, 40.0 mg), 2,3,5,6-tetrachloro-1,4-benzoquinone (TCBQ 54.5 mg), sodium carbonate (122.3 mg), and n-butanol (2 mL). The mixture was frozen to 78 K in a liquid nitrogen bath and evacuated for 10 min, followed by warming to room temperature after sealing. After another two freeze-pump-thaw cycles, the mixture was heated to 120° C. and reacted for 72 h. The crude produce was filtered and washed with water, dimethylformamide, tetrahydrofuran, and dichloromethane to yield a brown solid (49.4 mg, 79.5%).

### Example 2

### Synthesis of benzo[5,6][1,4]dithiino[2,3-b]thianthrene-6,13-dione (Model Compound)

Benzene-1,2-dithiol (100 mg, 0.7 mmol), 2,3,5,6-tetrachloro-1,4-benzoquinone (72 mg, 0.3 mmol), and potassium carbonate (249 mg, 1.8 mmol) were charged in a flask. After evacuating and filling with argon, N,N′-dimethylformamide (5 mL) was added and stirred at 90° C. for 24 h. The product was purified by column chromatography to give a red-brown solid (81 mg, 70%).

### Example 3

### Characterization of BHT-BQ-COF and the Model Compound

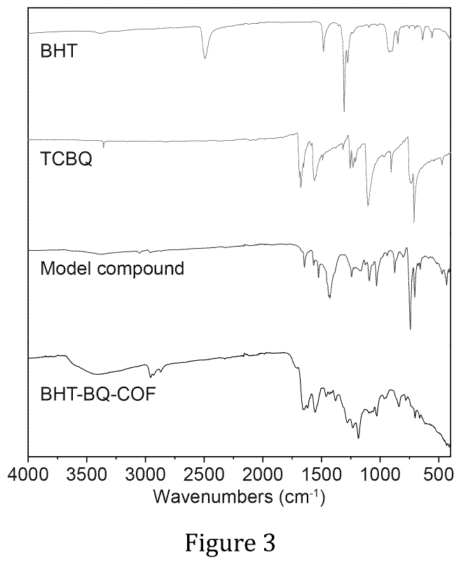

To verify the conformation of the dithioether linkage in BHT-BQ-COF, Fourier transform infrared (FTIR) spectra of the precursors of BHT-BQ-COF (i.e., 1,2,3,4,5,6-benzenehexathiol (BHT) and 2,3,5,6-tetrachloro-1,4-benzoquinone (TCBQ)), the BHT-BQ-COF of Example 1, and the model compound of Example 2 were obtained. FTIR spectra were recorded on a PerkinElmer Spectrum Two FTIR Spectrometer. As shown in FIG. 3, the disappearance of the —S—H bonds (2500 cm⁻¹) of BHT in BHT-BQ-COF indicates the fully conversion of the precursors after the coupling reaction. The appearance of the —C—S bonds (703 cm⁻¹ and 664 cm⁻¹) and ══C—S—C bonds (1233 cm⁻¹ and 1028 cm⁻¹) of BHT-BQ-COF indicates the formation of the dithioether linkage, which are in accordance with the peaks of the model compound at 705 cm⁻¹, 658 cm⁻¹, 1243 cm⁻¹, and 1030 cm⁻¹, respectively.

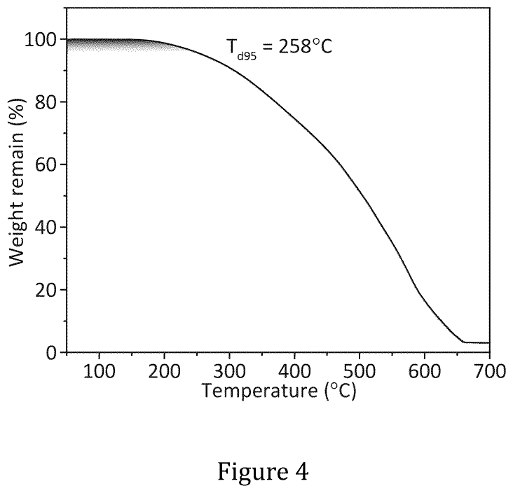

The thermal stability of BHT-BQ-COF was measured on a PerkinElmer STA 6000 using ceramic pan as the container. As shown by the thermogravimetric analysis in FIG. 4, more than 90% of the initial weight can be remained when the temperature reaches 230° C. The apparent drop before 100° C. can be ascribed to the moisture adsorbed by BHT-BQ-COF. This demonstrates the good thermal stability of BHT-BQ-COF.

### Example 4

### Fabrication of a BHT-BQ-COF-Based Cathode

BHT-BQ-COF, Ketjen Black, and polyvinylidene fluoride (PVDF) were mixed in N-methyl-2-pyrrolidone (NMP) with a mass ratio of 5:4:1 to form a slurry. The obtained slurry was pasted onto an Al foil and dried at 80° C. for 12 h in a vacuum oven.

### Example 5

### Fabrication of a Half SIB Coin Cell

A sodium disk was first pressed to the stainless steel spacer. Then an o-ring was placed on the smaller case and pressed against the case. Next, a spring, the assembled stainless steel spacer and sodium disk were placed on the o-ring sequentially, with the sodium disk facing upwards. After that, a separator (glass fiber) was placed on top of the sodium disk as centered as possible. 200 μL of 1M sodium hexafluorophosphate (NaPF₆) in diglyme as the electrolyte was then dropped onto the separator. Next, the BHT-BQ-COF-based cathode obtained in Example 4 was placed on top, with the cast film facing the sodium disk and centered as much as possible with the sodium disk to avoid uneven current densities. Finally the larger case was placed on top and the coin cell was packed with 0.8 kPa pressure.

### Example 6

### Electrochemical Performance of the Half SIB Coin Cell

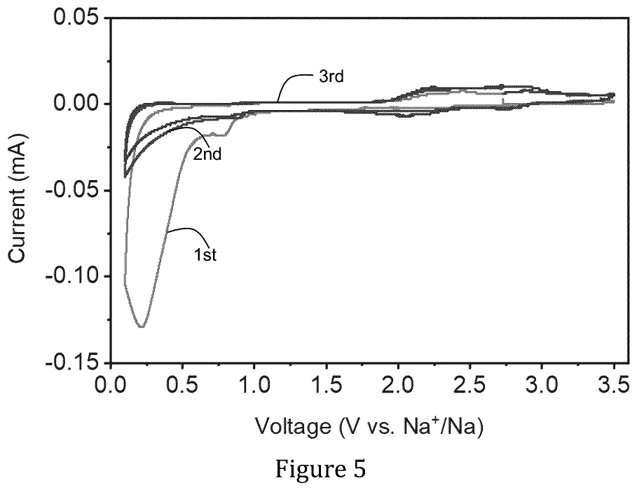

Coin cells of CR2032 were first assembled in an argon-filled glovebox (O₂≤3.1 ppm, H₂O≤3.1 ppm). These coin cells were used to investigate the electrochemical performance of the half SIB coin cell obtained in Example 5, with Na foil as the counter electrode (Na//BHT-BQ-COF SIB). FIG. 5 shows the cyclic voltammetry (CV) curves of the half SIB coin cell. The CV tests were carried out on a Zahner electrochemical workstation with a voltage range of 0.1 V to 3.5 V and a scan rate of 0.1 mV s⁻¹. As shown in FIG. 5, the broad peak located at 0.1 V to 1.0 V during the first anodic process is attributed to the formation of solid electrolyte interphase (SEI) on the electrode surface. The redox peaks located at 2.23/2.13 and 2.82/2.74 demonstrate the reversible redox reaction of BHT-BQ-COF electrode.

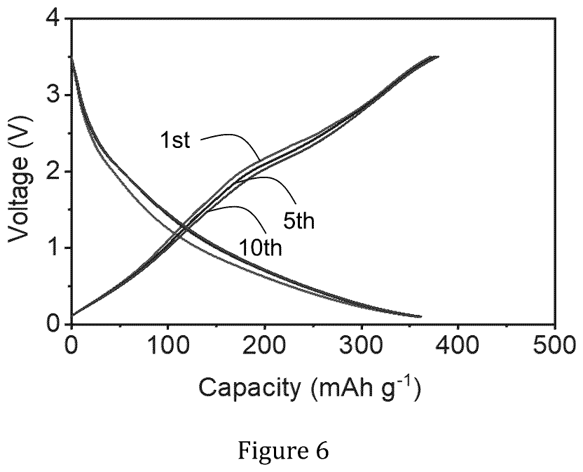

FIG. 6 shows the voltage-capacity curves of the BHT-BQ-COF-based electrode in the sodium-ion battery at the first, fifth, and tenth cycles. They demonstrate the Na storage performance of the BHT-BQ-COF-based electrode in SIBs with a high reversible capacity of about 350 mAh g⁻¹ at a current density of 20 mA g⁻¹, indicating the abundant redox active sites of BHT-BQ-COF.

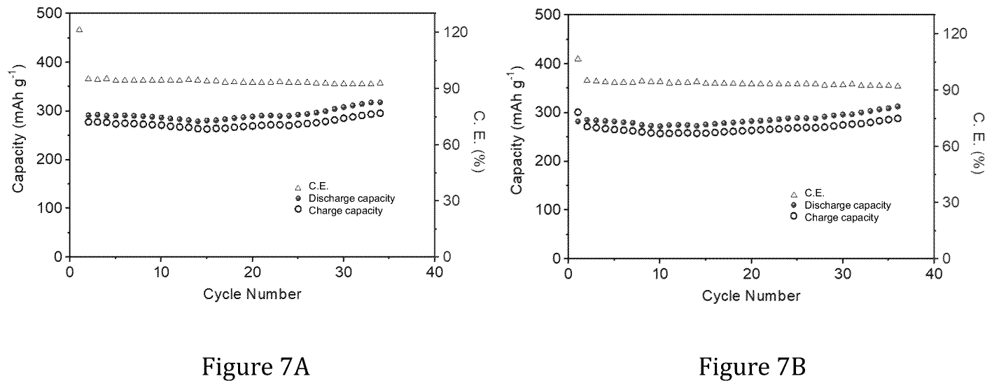

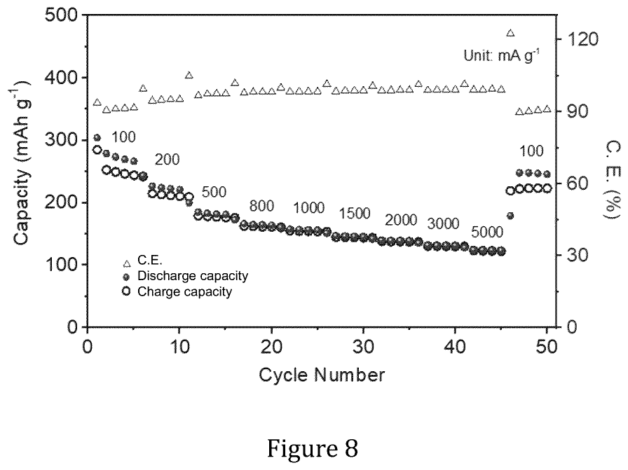

Galvanostatic charge/discharge measurements were performed on a MAC COR battery cycler at current densities of 100, 200, 400, 500, 800, 1000, 1500, 2000, 3000 and 5000 mA g⁻¹. FIGS. 7A and 7B show the high cycling stability with reversible capacities of 290 mAh g⁻¹ and 282 mAh g⁻¹ at the current densities of 200 mA g⁻¹ and 400 mA g⁻¹, respectively, which is associated with the stable structure of BHT-BQ-COF. FIG. 8 shows the superior rate capability of the BHT-BQ-C OF-based electrode at a wide current density range of 100 mA g⁻¹ to 5000 mA g⁻¹ with a voltage range of 0.1 V to 3.5 V, showing the fast Na⁺ diffusion kinetics of the electrode.

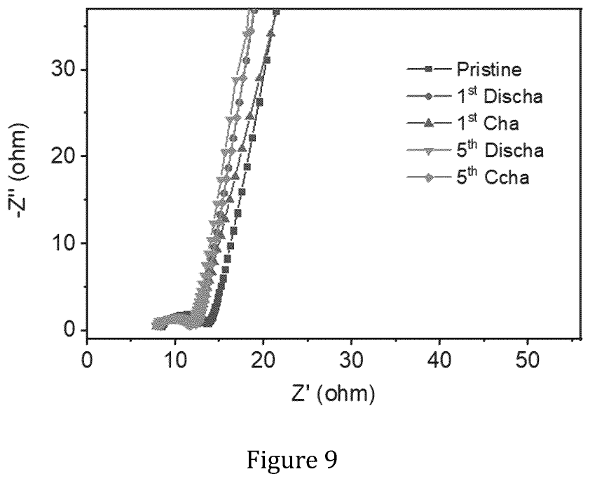

FIG. 9 show the electrochemical impedance spectroscopy (EIS) curves of the cell of Na//BHT-BQ-COF-based electrode at different charge and discharge states with a wide frequency range from 100 kHz to 100 mHz. Electrochemical impedance spectroscopy (EIS) was obtained by applying a sine wave with an amplitude of 10 mV in the frequency range from 100 kHz to 100 mHz on Solartron 1400 electrochemical workstation. As shown in FIG. 9, the charge-transfer resistance (Ret) corresponds to the semicircle at high frequencies. The small R_{ct} in the cell of Na//BHT-BQ-COF remains constant after the charge and discharge processes, which manifests fast reaction kinetics, specifically Na⁺ diffusion kinetics, on the electrode surface and enables excellent rate performance of BHT-BQ-C OF-based electrodes.

The above embodiments and examples provide a dithioether-linked COF with a much better stability in electrolyte than typical organic small molecules. Its porous and rigid structure, easiness of modification and insolubility allow it to be a promising organic cathode material for rechargeable sodium-ion batteries. High performance such as high capacity and excellent cyclic stability and reversibility can be simultaneously achieved when combining proper electrolyte and additives that, e.g., provide multiple redox-reactive sites. The COF also provides a large specific area that is essential for efficient ion insertion and extraction.

It will be appreciated by persons skilled in the art that numerous variations and/or modifications may be made to the invention as shown in the specific embodiments without departing from the spirit or scope of the invention as broadly described. The described embodiments of the invention should therefore be considered in all respects as illustrative, not restrictive.

1. A covalent organic framework, comprising:
a plurality of aromatic moieties each linked by at least one thioether linkage.

2. The covalent organic framework of claim 1, wherein each of the plurality of aromatic moieties is linked by two thioether linkages.

3. The covalent organic framework of claim 1, wherein each of the plurality of aromatic moieties comprises at least one carbonyl group.

4. The covalent organic framework of claim 2, wherein each of the plurality of aromatic moieties comprises two carbonyl groups.

5. The covalent organic framework of claim 3, wherein each of the plurality of aromatic moieties is a benzoquinone moiety.

6. The covalent organic framework of claim 1 comprising a plurality of dithioether-linked benzoquinone moieties.

7. An energy storage device, comprising a cell with an electrode comprising the covalent organic framework of claim 1.

8. The energy storage device of claim 7, wherein the covalent organic framework further comprises a plurality of redox active sites for facilitating diffusion of ions of the energy storage device.

9. The energy storage device of claim 8, wherein the plurality of redox active sites comprise a sulfur atom of the thioether linkages.

10. The energy storage device of claim 8, wherein each of the plurality of aromatic moieties comprises at least one carbonyl group, and wherein the plurality of redox active sites comprise the carbonyl groups.

11. The energy storage device of claim 7, wherein the electrode is a cathode.

12. The energy storage device of claim 7, wherein the cell is rechargeable.

13. The energy storage device of claim 7, wherein the cell comprises a sodium-ion battery or a lithium-ion battery.

14. The energy storage device of claim 7 takes the form of a coin cell.

15. A method of preparing the covalent organic framework of claim 1, comprising the steps of:
mixing an aromatic compound and a thiol to form a mixture;
degassing the mixture; and
heating the degassed mixture.

16. The method of claim 15, wherein the aromatic compound comprises a quinone.

17. The method of claim 16, wherein the quinone is a halogen-substituted quinone.

18. The method of claim 17, wherein the halogen-substituted quinone is 2,3,5,6-tetrachloro-1,4-benzoquinone or 2,3,5,6-tetrafluoro-1,4-benzoquinone.

19. The method of claim 15, wherein the thiol is an aromatic thiol.

20. The method of claim 19, wherein the aromatic thiol is 1,2,3,4,5,6-benzenehexathiol.

21. The method of claim 15, wherein the step of mixing comprises mixing a base and a solvent with the aromatic compound and the thiol to form the mixture, the base comprising any one of sodium carbonate, potassium carbonate, cesium carbonate, sodium hydroxide, sodium methoxide, and N,N-diethylethanamine, and the solvent comprising any one of 1,2-dichlorobenzene, n-butanol, 1,4-dioxane, mesitylene, N-methylpyrrolidone, N,N-dimethylacetamide, or a mixture thereof.

22. The method of claim 15, further including sonicating the mixture to form a homogeneous mixture.

23. The method of claim 15, wherein the step of degassing comprises subjecting the mixture to at least one freeze-pump-thaw cycle.
